(19) United States
(12) Patent Application Publication
(10) Pub. No.: US 2004/0002137 A1
(43) Pub. Date: Jan. 1, 2004

(54) Human tissue urokinase type plasminogen activator production

(75) Inventors: Paul Porwen Hung, Bryn Mawr, PA (US); Bryan T. H. Wu, Taipei (TW)

Correspondence Address: FISH & RICHARDSON PC, 225 FRANKLIN ST, BOSTON, MA, 02110, US

(73) Assignee: Global Biotech, Inc. a Taiwan corporation

(21) Appl. No.: 10/401,077

(22) Filed: Mar. 27, 2003

Related U.S. Application Data
(60) Provisional application No. 60/371,013, filed on Apr. 9, 2002.

(51) Int. Cl. C12N 9/06; C12N 9/72; C07H 21/04; C12P 21/02; C12N 5/06; C12N 15/86

(52) U.S. Cl. 435/69.1; 435/235.1; 435/191; 435/215; 435/325; 536/23.2; 435/456

(57) ABSTRACT

This invention features a nucleic acid expression vector that includes a bicistronic coding unit that comprises a first segment that encodes a human tissue urokinase plasminogen activator protein and a second segment that encodes an amplifiable dominant selectable marker (e.g., dihydrofolate reductase); and a promoter (e.g., a cytomegalovirus promoter) operably linked to the bicistronic coding unit.

### RELATED APPLICATIONS

This application claims priority to U.S. Provisional Application Serial No. 60/371,013 filed Apr. 9, 2002, the contents of which are incorporated herein by reference.

### BACKGROUND

Plasminogen activators are a class of serine proteases that convert plasminogen into plasmin. Plasmin degrades the fibrin matrix of blood clots, thereby restoring the hemodynamic condition of an open vascular system after an internal vascular accident has produced thrombosis or thromboembolism. Vascular disease states, which involve partial or total blockage of blood vessels and are amenable to treatment with plasminogen activators, include stroke, pulmonary embolism, myocardial infarction, as well as deep vein and peripheral artery obstructions.

There are two immunologically distinct types of plasminogen activators found in human plasma and other body fluids: the urokinase-type plasminogen activator (UPA; Mr, 55,000) and the tissue-type plasminogen activator (TPA; Mr, 68,000). The activity of the tissue-type plasminogen activator is potentiated by fibrin. This enzyme acts at the site of a thrombus and demonstrates a higher affinity for fibrin than does the urokinase-type plasminogen activator (Haylaeris et al. (1982) J. Biol. Chem. 257: 2912). In fact, a recombinant form of the tissue-type plasminogen activator, brought to market by Genentech (European Patent No. 93,619), has been in use for medicine for many years. But this recombinant form is short-lived (half life≈4 min) in plasma. Although a second generation of the recombinant tissue-type plasminogen activator has been engineered, it still falls short of the need for new generation of the tissue-type plasminogen activator with a longer half life.

Human tissue urokinase plasminogen activator (HTU-PA) is a hybrid protein of the human tissue plasminogen activator and the human urokinase plasminogen activator. The hybrid protein not only is fibrinolytically active but also offers the advantages of increased stability, increased binding affinity for fibrin, and improved half-life in vivo (half life≈30 min), compared to either of the human tissue plasminogen activator or the human urokinase plasminogen. See, e.g., Kalyan et al. (1988) Gene 68(2): 205-12; Weinheimer et al. (1991) Circulation. 83(4): 1429-36; Hung et al. (1990) Adv Exp Med Biol. 281: 201-8; and Lee et al. (1988) J Biol Chem. 263(6): 2917-24.

High level expression of plasminogen activators has been studied. Two expression systems include the mouse mammary tumor virus promoter and BPV vector p504 (U.S. Pat. No. 4,916,071) and pTRB1/2 (Samanta & Kim (1991) J. Biotech 20: 1-16), and express the tissue-type plasminogen activator in mouse C-127 cells at a very high level (10-20 μg/mL/million cells). In these systems, human tissue urokinase plasminogen activator can also be expressed at the similar level (Lee et al. supra). Even both systems offer a Cd++ regulatable expression, it is not needed as both systems function constitutively. Both, however, suffer from cell detachment during production of plasminogen activators. Although both systems offer collagen-based micro-carrier based beads as a remedy, cell integrity and consistency for a successful manufacturing remains a problem.

Thus, it is desirable to develop another method that can produce the human tissue urokinase plasminogen activator efficiently.

### SUMMARY

This invention is based on the discovery of a nucleic acid vector capable of high-level expression of human tissue urokinase plasminogen activator.

In one aspect, this invention features a nucleic acid expression vector that includes a bicistronic coding unit that comprises a first segment that encodes a human tissue urokinase plasminogen activator protein and a second segment that encodes an amplifiable dominant selectable marker (e.g., dihydrofolate reductase); and a promoter (e.g., a cytomegalovirtis promoter) operably linked to the bicistronic coding unit. The human tissue urokinase plasminogen activator protein, as used herein, includes SEQ ID NO:1 (which encodes SEQ ID NO:2), or equivalents of SEQ ID NO:1, having a percent identity of at least 80% (e.g., 90%, 95% or 99%) and possessing essentially the same fibrinolytic activity (+20%), e.g., see Lee et al. supra. The just-described sequences are shown below.

| | |
|---|---|
| atggatgcaatgaagagagggctctgctgtgtgctgctgctgtgtggagcagtcttcgtt | (SEQ ID NO:1) |
| M D A M K R G L C C V L L L C G A V F V | (SEQ ID NO:2) |
| tcgcccagccaggaaatccatgcccgattcagaagaggagccagatcttaccaagtgatc | |
| S P S Q E I H A R F R R G A R S Y Q V I | |
| tgcagagatgaaaaaacgcagatgatataccagcaacatcagtcatggctgcgccctgtg | |
| C R D E K T Q M I Y Q Q H Q S W L R P V | |
| ctcagaagcaaccgggtggaatattgctggtgcaacagtggcagggcacagtgccactca | |
| L R S N R V E Y C W C N S G R A Q C H S | |
| gtgcctgtcaaaagttgcagcgagccaaggtgtttcaacgggggcacctgccagcaggcc | |
| V P V K S C S E P R C F N G G T C Q Q A | |
| ctgtacttctcagatttcgtgtgccagtgccccgaaggatttgctgggaagtgctgtgaa | |
| L Y F S D F V C Q C P E G F A G K C C E | |
| atagataccagggccacgtgctatgaggggaatggtcacttttaccgaggaaaggccagc | |
| I D T R A T C Y E G N G H F Y R G K A S | |
| actgacaccatgggccggccctgcctgccctggaactctgccactgtccttcagcaaacg | |
| T D T M G R P C L P W N S A T V L Q Q T | |
| taccatgcccacagatctgatgctcttcagctgggcctggggaaacataattactgcagg | |
| Y H A H R S D A L Q L G L G K H N Y C R | |
| aacccagacaaccggaggcgaccctggtgctatgtgcaggtgggcctaaagccgcttgtc | |
| N P D N R R R P W C Y V Q V G L K P L V | |
| caagagtgcatggtgcatgactgcagcgagggcaactccgactgctacgaggaccagggc | |
| Q E C M V H D C S E G N S D C Y E D Q G | |
| atcagctacaggggcacgtggagcacagcggagagtggcgccgagtgcaccaactggaac | |
| I S Y R G T W S T A E S G A S C T N W N | |
| agcagcgcgttggcccagaagccctacagcgggcggaggccagacgccatcaggctgggc | |
| S S A L A Q K P Y S G R R P D A I R L G | |
| ctggggaaccacaactactgcagaaacccagatcgagactcaaagccctggtgctacgtc | |
| L G N H N Y C R N P D R D S K P W C Y V | |
| tttaaggcggggaagtacagctcagagttctgcagcacccctgcctgctctgagggaaac | |
| F K A G K Y S S E F C S T P A C S E G N | |
| agtgactgctactttgggaatgggtcagcctaccgtggcacgcacagcctcaccgagtcg | |
| S D C Y F G N G S A Y R G T H S L T E S | |
| ggtgcctcctgcctcccgtggaattccatgatcctgataggcaaggtttacacagcacag | |
| G A S C L P W N S M I L I G K V Y T A Q | |
| aaccccagtgcccaggcactgggcctgggcaaacataattactgccggaatcctgatggg | |
| N P S A Q A L G L G K H N Y C R N P D G | |
| gatgccaagccctggtgccacgtgctgaagaaccgcaggctgacgtgggagtactgtgat | |
| D A K P W C H V L K N R R L T W E Y C D | |
| gtgccctcctgctccacctgcggcctgagacagtacagccagcctcagtttcgcatcaaa | |
| V P S C S T C G L R Q Y S Q P Q F R I K | |
| ggagggctcttcgccgacatcgcctcccacccctggcaggctgccatctttgccaagcac | |
| G G L F A D I A S H P W Q A A I F A K H | |
| aggaggtcgcccggagagcggttcctgtgcgggggcatactcatcagctcctgctggatt | |
| R R S P G E R F L C G G I L I S S C W I | |
| ctctctgccgcccactgcttccaggagaggtttccgccccaccacctgacggtgatcttg | |
| L S A A H C F Q E R F P P H H L T V I L | |
| ggcagaacataccgggtggtccctggcgaggaggagcagaaatttgaagtcgaaaaatac | |
| G R T Y R V V P G E E E Q K F E V E K Y | |
| attgtccataaggaattcgatgatgacacttacgacaatgacattgcgctgctgcagctg | |
| I V H K E F D D D T Y D N D I A L L Q L | |
| aaatcggattcgtcccgctgtgcccaggagagcacgcgtggtccgcactgtgtgccttccc | |
| K S D S S R C A Q E S S V V R T V C L P | |
| ccggcggacctgcagctgccggactggacggagtgtgagctctccggctacggcaagcat | |
| P A D L Q L P D W T E C E L S G Y G K H | |
| gaggccttgtctcctttctattcggagcggctgaaggaggctcatgtcagactgtaccca | |
| E A L S P F Y S E R L K E A H V R L Y P | |
| tccagccgctgcacatcacaacatttacttaacagaacagtcaccgacaacatgctgtgt | |
| S S R C T S Q H L L N R T V T D N M L C | |
| gctggagacactcggagcggcgggccccaggcaaacttgcacgacgcctgccagggcgat | |
| A G D T R S G G P Q A N L H D A C Q G D | |
| tcgggaggccccctggtgtgtctgaacgatggccgcatgactttggtgggcatcatcagc | |
| S G G P L V C L N D G R M T L V G I I S | |
| tggggcctgggctgtggacagaaggatgtcccgggtgtgtacacaaaggttaccaactac | |
| W G L G C G Q K D V P G V Y T K V T N Y | |
| ctagactggattcgtgacaacatgcgaccgtgaccaggaacacccgactcctcaaaagca | |
| L D W I R D N N R P - | |
| aatgagatccggatcc | |

In another aspect, this invention features a nucleic acid vector that includes a bicistronic coding unit that comprises a first segment that encodes a human tissue urokinase plasminogen activator protein comprising SEQ ID NO:1 and a second segment that encodes dihydrofolate reductase; and a cytomegalovirus promoter operably linked to the bicistronic coding unit.

In further another aspect, this invention features a mammalian CHO host cell. The mammalian host cell includes a nucleic acid which comprises a bicistronic coding unit that comprises a first segment that encodes a human tissue urokinase plasminogen activator protein and a second segment that encodes an amplifiable dominant selectable marker (e.g., dihydrofolate reductase); and a promoter (e.g., a cytomegalovirus promoter) operably linked to the bicistronic coding unit. The CHO host cell can be a DG44 cell or a DXB11 cell, and can produce at least 0.5 μg/million cells the human tissue urokinase plasminogen activator protein in a suspension culture.

Also within the scope of this invention is a method for producing a recombinant human tissue urokinase plasminogen activator protein. The method includes culturing a mammalian host cell of described above in a medium under conditions promoting expression of the bicistronic coding unit and production of the human tissue urokinase plasminogen activator protein; and isolating the human tissue urokinase plasminogen activator protein from the cultured mammalian host cell or the medium.

As used herein, the term “vector” refers to a nucleic acid molecule capable of transporting another nucleic acid to which it has been linked and can include a plasmid, cosmid or viral vector. The vector can be capable of autonomous replication or it can integrate into a host DNA.

The term “bicistronic coding unit” refers to a region that can be transcribed to produce a transcript for a plurality of reading frames. An exemplary bicistronic unit includes segments that encode a human tissue urokinase plasminogen activator protein and an amplifiable dominant selectable marker, that are expressed simultaneously from the same promoter so that virtually all transfected cells express both the human tissue urokinase plasminogen activator protein and the selection marker.

The “percent identity” of two amino acid sequences is determined using the algorithm of Karlin and Altschul (1990, Proc. Natl. Acad. Sci. USA 87: 2264-2268), modified as in Karlin and Altschul (1993, Proc. Natl. Acad. Sci. USA 90: 5873-5877). Such an algorithm is incorporated into the NBLAST and XBLAST programs of Altschul et al. (1990, J. Mol. Biol. 215: 403-410). BLAST nucleotide searches are performed with the NBLAST program, score=100, wordlength=12. BLAST protein searches are performed with the XBLAST program, score=50, wordlength=3. Where gaps exist between two sequences, Gapped BLAST is utilized as described in Altschul et al. (1997, Nucleic Acids Res. 25: 3389-3402). When utilizing BLAST and Gapped BLAST programs, the default parameters of the respective programs (e.g., XBLAST and NBLAST) are used. Sequences that are at least 90%, 95%, 98%, or 99% identical to SEQ ID NO: 1 are contemplated.

Other features, objects, and advantages of the invention will be apparent from the description and from the claims.

### DETAILED DESCRIPTION

The invention relates to a nucleic acid vector capable of high-level expression of a human tissue urokinase plasminogen activator protein. An example of the human tissue urokinase plasminogen activator protein is SEQ ID NO: 1 (see above).

A nucleic acid vector of this invention includes synthetic or cDNA-derived DNA fragments encoding a human tissue urokinase plasminogen activator protein and a amplifiable dominant selectable marker, operably linked to one or more regulatory sequences. The regulatory sequences control expression of an mRNA transcript that includes two reading frames, i.e., the transcript is bi-cistronic. The 5′ cistron can encode the tissue urokinase plasminogen activator protein. Alternatively, the 5′ cistron can include the selectable marker.

The regulatory sequences include promoters, enhancers and other expression control elements (e.g., polyadenylation signals). Regulatory sequences also include those which direct constitutive expression of a nucleotide sequence, as well as tissue-specific regulatory and/or inducible sequences. DNA regions are operably linked when they are functionally related to each other. For example, a promoter is operably linked to a coding sequence if it controls the transcription of the sequence. The just-described expression vector is designed for expression of a human tissue urokinase plasminogen activator protein in a mammalian host cell. In such a host cell, the expression vector's control functions can be provided, e.g., by viral regulatory elements. For example, commonly used promoters are derived from polyoma, Adenovirus 2, cytomegalovirus, and Simian Virus 40.

In some embodiments, a nucleic acid vector of this invention includes a segment encoding a human tissue urokinase plasminogen activator protein comprising SEQ ID NO:1 and a segment encoding dihydrofolate reductase; and a cytomegaloviris promoter operably linked to the coding units.

A vector nucleic acid can be introduced into host cells via conventional transformation or transfection techniques, which are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell of the invention can be used to produce (i.e., express) a human tissue urokinase plasminogen activator protein. Accordingly, the invention further provides methods for producing a human tissue urokinase plasminogen activator protein using a host mammalian cell. The method includes culturing the host cell (into which a recombinant expression vector encoding a human tissue urokinase plasminogen activator protein has been introduced) in a suitable medium such that a human tissue urokinase plasminogen activator protein is produced. The method further includes isolating a human tissue urokinase plasminogen activator protein from the medium or the host cell. The isolated protein can be formulated as a pharmaceutical composition, e.g., for administration to a subject.

Various mammalian cell culture systems can be employed to express a human tissue urokinase plasminogen activator protein. Examples of suitable mammalian host cell lines include CV-I/EBNA (ATCC CRL 10478), L cells, C127, 3T3, CHO (e.g., DG44 or DXB11), HeLa and BHK cell lines.

Useful amplifiable selectable markers for gene amplification in drug-resistant cells have been described in, e.g., Kaufman (1988, Meth. Enzymology 185: 537), including DHFR-MTX resistance, P-glycoprotein and multiple drug resistance (MDR)-various lipophilic cytoxic agents (i.e., adriamycin, colchicine, vincristine), and adenosine deaminase (ADA)-Xyl-A or adenosine and 2′-deoxycoformycin. Protocols employing two dominant selectable markers have been described in Okayama and Berg (1985, Mol. Cell Biol 5: 1136).

Useful regulatory sequences, as described previously, can be included in plasmids used to transform mammalian cells. The transformation protocol chosen, and the sequences selected for use therein, will depend on the type of host cell used. Those of skill in the art are aware of numerous different protocol and host cells, and can select an appropriate system for expression of a human tissue urokinase plasminogen activator protein, based on the requirements of their cell culture systems.

Within the scope of this invention is a recombinant nucleic acid containing a bicistronic coding unit that includes a segment encoding a human tissue urokinase plasminogen activator protein, a segment encoding dihydrofolate reductase, and a cytomegalovirus promoter operably linked to it. When such a nucleic acid is introduced into a host cell (e.g., a mammalian CHO cell) for producing the human tissue urokinase plasminogen activator protein, great consistency is obtained. Unexpectedly, cell detachment during the production does not occur.

The produced human tissue urokinase plasminogen activator can then be purified by column chromatography or other techniques, if necessary. Purity can be readily measured by any appropriate method, for example, column chromatography, polyacryamide gel electrophoresis, high-pressure liquid chromatography analysis, or analysis of fibrinolytic activity.

A human tissue urokinase plasminogen activator protein can be used for preventing or treating conditions in which it is desired to produce local fibrinolytic activity via the plasminogen activation. The conditions include stroke, venous thrombosis, pulmonary embolism, or deep vein and peripheral artery obstruction. See Bang, N. U. (1989) Circulation 79: 1391-1392. The fibrinolytic activity of a human tissue urokinase plasminogen activator can be determined by a method described in, for example, U.S. Pat. No. 4,777,043. A pharmaceutical composition for treating the afore-mentioned conditions includes an effective amount of the human tissue urokinase plasminogen activator. The effective amount refers to that amount providing therapeutic effect on the treated subject. The effective amount will also vary, as recognized by those skilled in the art, depending on the excipient usage, route of administration, and the possibility of co-usage with other therapeutic treatment.

The pharmaceutical composition can be used for routes of administration utilizing conventional methods. For example, it can be administered via parenteral route including various injection or infusion techniques. A sterile injectable preparation, for example, a sterile injectable aqueous solution, is formulated according to techniques known in the art using a suitable diluent, e.g., sterile water. A desired solution concentration may vary depending on the particular use envisioned.

Without further elaboration, it is believed that one skilled in the art can, based on the above disclosure and the description below, utilize the present invention to its fullest extent. The following example is to be construed as merely illustrative of how one skilled in the art can practice the invention and are not limitative of the remainder of the disclosure in any way. Any publications cited in this disclosure are hereby incorporated by reference.

### EXAMPLE

Materials And Methods:

Expression Vector. Human tissue urokinase plasminogen activator (HTU-PA) was cloned into a vector (pCED) generated from commercially available pcDNA3 (Invitrogen) and pED. The HTU-PA open reading frame (ORF) was cloned by PCR using recombinant mouse C127 cells DNA as a template. The following primers containing Bam HI (Forward primer, ATTGGATCCAGCAATCATGGATGC) and Xba I (Reverse primer, TATTCTAGAGGCCTGGTCACGGTCTG) restriction sites, respectively were used in a PCR reaction. The amplification was done in 50 μL using the following conditions: 94° C. for 30 sec., 60° C. for 30 sec. and 72° C. for 90 sec. for 20 cycles and a final extension at 72° C. for 10 min. The PCR product was then digested with BamH I and Xba I restriction enzymes as per manufacturer's instructions (Boehringer-Mannheim) and the digested products were separated on a 1% low melting agarose gel. A fragment of about 2.0 Kb was identified and purified using QiaQuick™ gel extraction kit (Qiagen). The expression plasmid pcDNA3 was also digested with BamH I and Xba I and the digested products were separated on the gel. A desired fragment was purified as described above. The vector and the just-obtained fragments were then ligated and used to transform DH5a cells. The recombinant HTU-PA clone was identified by sizing and subsequently by restriction analysis. The plasmid DNA was purified using a Qiagen column as recommended by the supplier (Qiagen).

The pcDNA3 HTU-PA clone contains ORF of HTU-PA under the control of cytomegalovirus (CMV) promoter. The CMV promoter and the HTU-PA ORF was excised from the above plasmid using PvuI and XbaI and a ˜3.9 Kb fragment was separated on an agarose gel. The vector pED was also digested with PvuI and XbaI and the appropriate fragments were identified, separated on a gel, and purified, ligated and transformed according to the protocol described above. The recombinant plasmid was identified using restriction analysis of the plasmid.

The pED-HTU-PA plasmid now contains the CMV promoter driving a bi-cistronic region containing ORF of HTU-PA and dihydrofolate reductase (DHFR). This plasmid was named pCED. The EMC leader sequence, upstream of DHFR, aided in initiation of the ribosome so that both HTU-PA and DHFR can be translated at the same time. The pCED clone was used to establish the stable cell lines resistant to methotrexate.

CHO Cell Lines. DG44 is a double deletion mutant and contain no endogenous DHFR sequences. These cells require hypoxanthine (or adenosine), glycine and thymidine for growth and have a reversion frequency of less than 10⁻⁸. In DXB11 cells, one DHFR allele has been deleted whereas the other allele carries a single DHFR mis-sense mutation.

Growth Media. A vial each of DG44 and DXB11 cells kept frozen in liquid nitrogen was thawed at 37° C. and transferred to 100 mm dishes along with 10 mL of the growth media, DMEM F12 (Gibco), containing 1× glutamax (Gibco), Penicillin/Streptomycin (P/S)(Gibco) and 30 μM Thymidine (Sigma). DMEM F12 also contains nucleosides to support DHFR-cell lines.

Selection Media. Recombinant Cell lines transfected with DHFR+ plasmids were supported in the αMEM (Gibco) media containing 10% dialyzed FBS, 1× Glutamax (Gibco) and 1×P/S (Gibco). This media has no nucleosides and is for DHFR+ cells.

Transfection Protocol. A Confluent T75 flask containing CHO cells was trypsinized. Cells were counted and about 1×10⁶ cells were added to each well of a six well plate in 4 mL of the growth media. After an overnight growth in a CO₂ incubator (cell doubled to ˜2×10⁶ cells) at 37° C., the media was removed and after washing each wells with Dulbecco's PBS (Gibco), 0.5 mL of Gibco's OPTIMEM-1 (Gibco) was then added to each well. Meanwhile, two separate solution of 2 μg of plasmid DNA in 250 μL of OPTIMEM-1 and 10 μL of Lipofectamine 2000 (Gibco) reagent in 250 μL of OPTIMEM-1 mixed after 30 min. at room temperature were added to four of the six wells, the remaining two well (Control wells) received no DNA. The plate was returned to the CO₂ incubator and incubated at 37° C. for 5 hr. At this point, another 1 mL of the selection media was added and incubation continued overnight. Next morning, the media was removed and replaced with 2 mL of the selection media and cells were incubated for another two days.

Methotrexate (MTX) Selection Protocol. A 5 mM stock solution of MTX (Mr=454.4) was prepared by dissolving 22.7 mg of MTX in 10 mL of the complete selection media. This stock solution was used for 20 μM (40 μL/10 mL/100 mm dish), 80 μM (160 μL/dish), 200 μM (400 μL/dish), 400 μM (800 μL/dish), 800 μM (1.60 mL/dish) and 1600 μM (3.2 mL/dish) levels of MTX. A second stock of 50 μM MTX was made by a 100-fold solution dilution of the 5 mM solution in the selection buffer. The 50 μM stock was used to attain 0.02 μM (4 μL/dish), 0.08 μM (16 μL/dish), 0.32 μM (64 μL/dish), 1.28 μM (256 μL/dish) and 5 μM (1.024 mL/dish) MTX in the selection media (10 mL).

Fibrin-Agar Plate Assay. Typically, fibrin-agar reagents are prepared either in Tris-buffer (0.05 M Tris, pH=7.4, 0.15 M NaCl) or Dulbecco's phosphate buffer (Gibco). Both buffers were tried. The Tris buffer system was abandoned because the set agar was too transparent to be discriminating for the clear zones. The phosphate buffer provided a good translucent background but did not dissolve fibrinogen too well. This buffer system was therefore modified slightly to the use of Tris-buffer only for the fibrinogen. It worked nicely.

Secondly, incubation with TPA protease was typically done overnight. 48 hrs were better for giving chance to the weaker samples also to show up. A recombinant TPA continued to clear the zone way past the 48 hrs incubation period. For instance a zone size of as large as 30 mm can result from a 72 hrs incubation with 10 μL recombinant TPA at 1 μg/mL. It is also helpful to incubate fibrin-agar plate inside a plastic box along with a wet paper towels for humidity.

Thrombin (500 units/mL; use 36 units/25 mL PBS buffer, pH=7.0 for 5 plates, warm to 47° C. before use) was mixed with plasminogen (2.5 units/mL; use 1 unit (400 μL)/25 mL in PBS buffer, pH=7.0 for 5 plates, warm to 47° C. before use). To the mixture, ME Agarose (400 mg/75 mL PBS buffer, pH=7.0 at 47° C.) was added, then fibrinogen (275 mg/25 mL PBS buffer, pH=7.0 for 5 plates, warmed to 37° C. before use). The mixture was mixed well and maintained at 47° C. A 30 mL Falcon plate was added and the mixture was cooled down. Store plates saran wrapped at 5° C. and suck out plugs with a 3 mm gel punch and vacuum just before use. Add 10 μL of each test sample and a control (e.g. TPA, 1 μg/mL) to each well and incubate in a closed plastic box with a wet towel (to provide humidity for 24-48 hrs). A diameter of a lytic zone of fibrin was measured for each sample. For the standard TPA dilutions, the diameters of zones were plotted against activity units of dilutions, and a standard curve was obtained. Using the standard curve, the diameter of zone was used to determine the activity and the amount of a HTU-PA sample.

Results:

Transfection. Two transfections were carried out using DG44 cells described above. In both transfections, a two day initial selection was done in the complete α-MEM media (no nucleosides) alone to make sure that DG44 cells not expressing the DHFR gene would not survive. The second selection involved inhibition of DHFR expression in different concentrations of MTX. In the first transfection, starting with 0.02 μM MTX, cells reaching confluency in a given concentration of MTX were split 1:10 to the next concentration of MTX in sixteen-fold increments. In the second transfection, MTX concentrations were raised in four-fold increments and a higher split ratio (1:50) to ensure a slower but firmer selection pressure for nucleoside deprivation.

After an early selection (0.02-80 μM MTX), 13 out of 24 clones showed expression of HTU-PA. Clone 68, for example, grew rapidly and reached the split stage in 80 μM MTX. The expression levels reached after amplification in 0.02, 1.28 and 80 μM MTX were comparable (˜0.50 μg/mL/million cells by the Fibrin-agar plate assay, or 8 μg/mL/10 mini/million cells/mL and 20 μg/20 min/million cells/mL by the amidolytic assay). UPA (157 I.U.) was also used as a standard, gave a 14 mm diameter of a zone clearing, which was comparable to the zone size obtained from 1 μg/mL of the recombinant TPA standard.

For the second transfection, one clone expressed HTU-PA at a level of around 0.40 μg/mL/million cells. This clone maintained active expressions after exposure to 5, 20, 80 μM MTX.

Assays with clone 68 indicated that HTU-PA activity remained stable to repeated freeze thawing of the cells in the culture medium used to grow cells (Table 1). Likewise, for clone 68, HTU-PA actually remained stable in monolayer cultures over a period of time (Table 2).

Further Gene Amplification. Clone 68 was subjected to gene amplification in 200, 400 and 800 μM MTX at a 1:50 cell split ratio. As is shown in Table 3, cell growth was highly retarded in high concentrations of MTX. Half way at these levels, particularly in 800 μM MTX, the cell grew in much reduced numbers only to recover fully afterwards. The retardation in cell growth was proportional to MTX concentrations (800 μM>400 μM>200 μM>80 μM). Clone 68 also grew well in 1600 AM MTX, but the initial HTU-PA expression level was low.

Optimum Number of Spits to reach State of Confluency. Normally, MTX concentration up to 80 μM, three to four splits were sufficient by which the cells became confluent. But if the concentrations of MTX were higher than 80 μM, it took as long as 10 weeks for cells to start to grow well. At this moment, gene amplification, as judged by the relative zone clearing in the fibrin-agar assay, was high. For HTU-PA, the level of expression in 80 μM MTX was doubled for clone 68—approximates 1 μg/million cells.

Single Cell Cloning. Fifty cells of clone 12 in 80 μM MTX and clone 68 in 80 μM, 200 μM, 400 μM, and 800 μM MTX were plated out in 100 mm Falcon plates in α-MEM media with a media change every 8 days. Foci were transferred (˜4 wks) and a good growth phase was attained (100 mm dishes, 8 weeks). For clone 12, 5 out of the 22 foci transferred showed good growth and were transferred to 100 mm plates (subclones 12-3, -5, -6, -7, and -8). All of these subclones were active for HTU-PA expression but have not yet reached the confluency to know the real expression levels.

For clone 68, out of the 36 foci transferred to a 24 well plate (Plate A and B), six subclones (68-A-14, 68-A-15, 68-A-16, 68-A-17, 68-B-9 and 68-B-10) grew well and were transferred to 100 mm plates for further amplification. Of these subclones 68-A-14 and -15, and -16 grew very fast and were active for HTU-PA expression. Sublones 68-A-14, -15, and -16 were found to express HTU-PA at levels 50% higher than that of clone 68. Of the remaining subclones (68-B-9 and 1-0), subclone 68-10-B grew better and tested positive for HTU-PA expression. Because of the very aggressive growth characteristics of subclones 68-A-14, -15, -16 and B-10, they were split in 200 M MTX. Subclone 68-A-14 was recently split in 400 LM MTX.

Clone 68 in 200 μM MTX gave two foci, both of which were transferred to a six-well plate, one of subclones (68-200-1) grew well and was active for HTU-PA expression. The subclone was not fully confluent in 200 μM MTX. No foci developed in 400 and 800 μM MTX even after 10 weeks in culture and were therefore discarded.

Discussions. The expression vector, pCED, is driven constitutively by the CMV promoter which is regarded as a very strong promoter for mammalian gene expression. HTU-PA was cloned in pCED and the recombinant termed as pCEDHTU-PA. The level of HTU-PA expression obtained in 80 μM MTX is 0.50 μg/million cells.

Clone purification improved the expression by 33-50% (subclones 68-A-14-16 and B-10), as described above. CHO cells can be easily made up through higher growth characteristics, which can be grown to at least 10 million cells per mL in suspension cultures—a 10-fold growth advantage over the surface restricted, anchor-dependent monolayer C127 cells. See Lee et al. (1988) J Biol Chem. 263(6): 2917-24.

In summary, HTU-PA was cloned into a constitutive pCED expression vector, driven by the CMV promoter under DHFR selection. Two transfections were carried out with DG44 cells and one with DXB11 cells. The recombinant DG44 cells from the first transfection were selected in MTX concentrations sixteen-fold at each step after achieving confluency (0.02, 0.32, 5.0, 80 μM) up to 80 μM and then raised to 200, 400 and 800 μM for clone 68. At least two subclones showed expression of HTU-PA in comparable amounts (0.50 μg/million cells). Attempt to obtain a higher expression level through gene amplification by levels of DHFR higher than 80 μM (200, 400, and 800 μM) were found toxic to the cells, although the 200 μM level was tolerated. In 400 to 800 μM MTX, cells rounded up from nucleoside starvation but then recovered after about two months of exposure to these concentrations of MTX. The recovery means a good growth rate and the spread out morphology. These cells express HTU-PA at levels about 83% higher than that of clone 68 in 80 μM MTX. To further enhance expression levels, Clones 12 and 68 in 80 μM MTX and clone 68 in 200 LM MTX have been purified. These cells have shown additional 33-50% improvement for the expression of HTU-PA.

During the third attempt at gene amplification, concentrations of MTX were raised slowly (four-fold rather than 16-fold) at a higher split-cell ratio (1:50) at each step of confluency to ensure an increased pressure for nucleoside starvation. Out of 26 foci picked, 15 have been growing well but clones 1, 2, 9 and 19 have been amplified to 200 μM MTX. Among these, HTU-PA clone 9 grew faster than others showing a 50% improvement at the expression level in 1.28 μM MTX over clone 68 in 80 μM MTX from the first transfection.

Thus, at this time it appears, that after single cell cloning and exposure of these single cell clones to 800 μM MTX, the HTU-PA expression level was at least 0.5 μg/million cells.

TABLE 1
Effect of freeze-thawing on HTU-PA stability.

| Clone | MTX | | | | |
|---|---|---|---|---|---|
| name | (μM) | 1/7/00 | 1/16/01 | 1/18/00 | 1/31/00 |
| 68 | 0.08 | 4 | 5 | 7 | 5.5 |
| 68 | 1.28 | | 6 | 8 | 6 |
| 68 | 80 | | 5 | 4 | 4 |

TABLE 2
HTU-PA stability in monolayer culture.

| Cone name | MTX levels (uM) | Days In Culture | Zone size (mm) |
|---|---|---|---|
| 68 | 200 | 2 | 4.5 |
| 68 | 200 | 3 | 6.5 |
| 68 | 400 | 2 | 6.0 |
| 68 | 400 | 3 | 6.0 |

TABLE 3
Effect of high concentrations of MTX on HTU-PA
expression levels and optimum period for confluency post-split.

| | MTX | | |
|---|---|---|---|
| | Concentrations | Zone size (mm), | Zone size (mm), |
| Clone name | (μM) | 3 wks post split | 10 wks post split |
| 68 | 80 | 8 | 6 |
| 68 | 200 | 6 | 7 |
| 68 | 400 | 5 | 8 |
| 68 | 800 | 4 | 11 |

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof can make various changes and modifications of the invention to adapt it to various usages and conditions. Thus, other embodiments are also within the claims.

What is claimed is:

1. A nucleic acid expression vector comprising:
a bicistronic coding unit that comprises a first segment that encodes a human tissue urokinase plasminogen activator protein and a second segment that encodes an amplifiable dominant selectable marker; and
a promoter operably linked to the bicistronic coding unit.

2. The expression vector of claim 1, wherein the amplifiable dominant selectable marker is dihydrofolate reductase.

3. The expression vector of claim 2, wherein the human tissue urokinase plasminogen activator protein comprises SEQ ID NO:1.

4. The expression vector of claim 1, wherein the promoter is a cytomegalovirus promoter.

5. The expression vector of claim 4, wherein the human tissue urokinase plasminogen activator protein comprises SEQ ID NO:1.

6. The expression vector of claim 1, wherein the human tissue urokinase plasminogen activator protein comprises SEQ ID NO:1.

7. The expression vector of claim 6, wherein the amplifiable dominant selectable marker is dihydrofolate reductase.

8. The expression vector of claim 6, wherein the promoter is a cytomegalovirus promoter.

9. A nucleic acid expression vector comprising:
a bicistronic coding unit that comprises a first segment that encodes a human tissue urokinase plasminogen activator protein comprising SEQ ID NO:1 and a second segment that encodes dihydrofolate reductase; and
a cytomegalovirus promoter operably linked to the bicistronic coding unit.

10. A mammalian CHO host cell comprising a nucleic acid which comprises:
a bicistronic coding unit that comprises a first segment that encodes a human tissue urokinase plasminogen activator protein and a second segment that encodes an amplifiable dominant selectable marker; and
a promoter operably linked to the bicistronic coding unit.

11. The mammalian host cell of claim 10, wherein the amplifiable dominant selectable marker is dihydrofolate reductase.

12. The mammalian host cell of claim 10, wherein the promoter is a cytomegalovirus promoter.

13. The mammalian host cell of claim 10, wherein the human tissue urokinase plasminogen activator protein comprises SEQ ID NO:1.

14. The mammalian host cell of claim 13, wherein the amplifiable dominant selectable marker is dihydrofolate reductase.

15. The mammalian host cell of claim 14, wherein the promoter is a cytomegalovirus promoter.

16. A method for producing a recombinant human tissue urokinase plasminogen activator protein, comprising:
culturing a mammalian host cell of claim 10 in a medium under conditions promoting expression of the bicistronic coding unit and production of the human tissue urokinase plasminogen activator protein; and
isolating the human tissue urokinase plasminogen activator protein from the cultured mammalian host cell or the medium.

17. The method of claim 16, wherein the amplifiable dominant selectable marker is dihydrofolate reductase.

18. The method of claim 16, wherein the promoter is a cytomegalovirus promoter.

19. The method of claim 16, wherein the human tissue urokinase plasminogen activator protein comprises SEQ ID NO: 1.

20. The method of claim 19, wherein the amplifiable dominant selectable marker is dihydrofolate reductase.

21. The method of claim 20, wherein the promoter is a cytomegalovirus promoter.


SEQUENCE LISTING (4 sequences)

SEQ ID NO: 1 - protein, 650 aa, Homo sapiens
MDAMKRGLCCVLLLCGAVFVSPSQEIHARFRRGARSYQVICRDEKTQMIYQQHQSWLRPVLRSNRVEYCWCNSGRAQCHSVPVKSCSEPRCFNGGTCQQALYFSDFVCQCPEGFAGKCCEIDTRATCYEGNGHFYRGKASTDTMGRPCLPWNSATVLQQTYHAHRSDALQLGLGKHNYCRNPDNRRRPWCYVQVGLKPLVQECMVHDCSEGNSDCYEDQGISYRGTWSTAESGAECTNWNSSALAQKPYSGRRPDAIRLGLGNHNYCRNPDRDSKPWCYVFKAGKYSSEFCSTPACSEGNSDCYFGNGSAYRGTHSLTESGASCLPWNSMILIGKVYTAQNPSAQALGLGKHNYCRNPDGDAKPWCHVLKNRRLTWEYCDVPSCSTCGLRQYSQPQFRIKGGLFADIASHPWQAAIFAKHRRSPGERFLCGGILISSCWILSAAHCFQERFPPHHLTVILGRTYRVVPGEEEQKFEVEKYIVHKEFDDDTYDNDIALLQLKSDSSRCAQESSVVRTVCLPPADLQLPDWTECELSGYGKHEALSPFYSERLKEAHVRLYPSSRCTSQHLLNRTVTDNMLCAGDTRSGGPQANLHDACQGDSGGPLVCLNDGRMTLVGIISWGLGCGQKDVPGVYTKVTNYLDWIRDNMRP

SEQ ID NO: 2 - DNA, 1996 bp, Homo sapiens
  CDS (1)...(1950)
atggatgcaatgaagagagggctctgctgtgtgctgctgctgtgtggagcagtcttcgtttcgcccagccaggaaatccatgcccgattcagaagaggagccagatcttaccaagtgatctgcagagatgaaaaaacgcagatgatataccagcaacatcagtcatggctgcgccctgtgctcagaagcaaccgggtggaatattgctggtgcaacagtggcagggcacagtgccactcagtgcctgtcaaaagttgcagcgagccaaggtgtttcaacgggggcacctgccagcaggccctgtacttctcagatttcgtgtgccagtgccccgaaggatttgctgggaagtgctgtgaaatagataccagggccacgtgctatgaggggaatggtcacttttaccgaggaaaggccagcactgacaccatgggccggccctgcctgccctggaactctgccactgtccttcagcaaacgtaccatgcccacagatctgatgctcttcagctgggcctggggaaacataattactgcaggaacccagacaaccggaggcgaccctggtgctatgtgcaggtgggcctaaagccgcttgtccaagagtgcatggtgcatgactgcagcgagggcaactccgactgctacgaggaccagggcatcagctacaggggcacgtggagcacagcggagagtggcgccgagtgcaccaactggaacagcagcgcgttggcccagaagccctacagcgggcggaggccagacgccatcaggctgggcctggggaaccacaactactgcagaaacccagatcgagactcaaagccctggtgctacgtctttaaggcggggaagtacagctcagagttctgcagcacccctgcctgctctgagggaaacagtgactgctactttgggaatgggtcagcctaccgtggcacgcacagcctcaccgagtcgggtgcctcctgcctcccgtggaattccatgatcctgataggcaaggtttacacagcacagaaccccagtgcccaggcactgggcctgggcaaacataattactgccggaatcctgatggggatgccaagccctggtgccacgtgctgaagaaccgcaggctgacgtgggagtactgtgatgtgccctcctgctccacctgcggcctgagacagtacagccagcctcagtttcgcatcaaaggagggctcttcgccgacatcgcctcccacccctggcaggctgccatctttgccaagcacaggaggtcgcccggagagcggttcctgtgcgggggcatactcatcagctcctgctggattctctctgccgcccactgcttccaggagaggtttccgccccaccacctgacggtgatcttgggcagaacataccgggtggtccctggcgaggaggagcagaaatttgaagtcgaaaaatacattgtccataaggaattcgatgatgacacttacgacaatgacattgcgctgctgcagctgaaatcggattcgtcccgctgtgcccaggagagcagcgtggtccgcactgtgtgccttcccccggcggacctgcagctgccggactggacggagtgtgagctctccggctacggcaagcatgaggccttgtctcctttctattcggagcggctgaaggaggctcatgtcagactgtacccatccagccgctgcacatcacaacatttacttaacagaacagtcaccgacaacatgctgtgtgctggagacactcggagcggcgggccccaggcaaacttgcacgacgcctgccagggcgattcgggaggccccctggtgtgtctgaacgatggccgcatgactttggtgggcatcatcagctggggcctgggctgtggacagaaggatgtcccgggtgtgtacacaaaggttaccaactacctagactggattcgtgacaacatgcgaccgtgaccaggaacacccgactcctcaaaagcaaatgagatccggatcc

SEQ ID NO: 3 - DNA, 24 bp, Artificial Sequence
  primer
attggatccagcaatcatggatgc

SEQ ID NO: 4 - DNA, 26 bp, Artificial Sequence
  primer
tattctagaggcctggtcacggtctg